(19) United States
(12) Patent Application Publication
(10) Pub. No.: US 2018/0005727 A1
(43) Pub. Date: Jan. 4, 2018

(54) APPARATUS CONTAINING A DIELECTRIC INSULATION GAS COMPRISING AN ORGANOFLUORINE COMPOUND

(71) Applicant: ABB Schweiz AG, Baden (CH)

(72) Inventors: Anna Di-Gianni, Dattwil (CH); Mariya Porus, Zurich (CH)

(21) Appl. No.: 15/649,421

(22) Filed: Jul. 13, 2017

Related U.S. Application Data
(63) Continuation of application No. PCT/EP2016/050545, filed on Jan. 13, 2016.

(30) Foreign Application Priority Data
Jan. 13, 2015 (EP) PCT/EP2015/050450

(51) Int. Cl. H01B 3/56; C01D 15/04; C07C 255/10; H02B 13/055

(52) U.S. Cl. CPC H01B 3/56; C07C 255/10; C01D 15/04; H02B 13/055

(57) ABSTRACT

The present invention relates to an apparatus for the generation, transmission, distribution and/or usage of electrical energy. The apparatus comprising a housing enclosing an insulating space and an electrical component arranged in the insulating space. The insulating space containing a dielectric insulation gas comprising an organofluorine compound. The apparatus further comprises a desiccant arranged such as to come into contact with the insulation gas. The desiccant contains or essentially consists of lithium bromide.

The present invention relates to an apparatus for the generation, transmission, distribution and/or usage of electrical energy according to the preamble of independent claim 1. The invention further relates to a method for providing a desiccant to such an apparatus.

Dielectric insulation media in liquid or gaseous state are conventionally applied for the insulation of an electrical component in a wide variety of apparatuses, such as for example switchgears, gas-insulated substations (GIS), gas insulated lines (GIL), or transformers.

In medium or high voltage metal-encapsulated switchgears, for example, the electrical component is arranged in a gas-tight housing, which defines an insulating space, said insulation space comprising an insulation gas and separating the housing from the electrical component, thus preventing electrical current to pass through the insulation space. For interrupting nominal or fault current in high voltage switchgear, the insulating gas further functions as an arc extinction gas.

Recently, the use of organofluorine compounds in an insulating gas has been suggested. Specifically, WO-A-2010/142346 discloses a dielectric insulation medium comprising a fluoroketone having from 4 to 12 carbon atoms. Furthermore, WO-A-2012/080246 discloses a fluoroketone containing exactly 5 carbon atoms (hereinafter referred to as “C5K”) in a mixture with a dielectric insulation gas component different from said C5K to be particularly preferred. A dielectric insulation medium comprising a hydrofluoromonoether has been disclosed in WO-A-2012/080222.

Both groups of compounds have been shown to have high insulation capabilities, in particular a high dielectric strength, as well as high arc extinction capabilities. At the same time, they have a very low Global Warming Potential (GWP) and very low toxicity. The combination of these characteristics renders these organofluorine compounds highly suitable as a substitute for SF₆ (sulphur hexafluoride), which has commonly been used as a dielectric insulation medium, but which is known to have a high GWP.

FR 2 965 120 discloses a circuit breaker comprising a dielectric insulation gas and containing a fluoroketone, which is partially liquid and partially gaseous, and comprising means for absorbing molecular species which are formed after ionisation of the fluoroketone in an arc.

However, in particular during e.g. a switching operation, which is accompanied by a high temperature increase in the insulation space, organofluorine compounds can be subject to decomposition. The formation of decomposition products can also be due to partial discharge and can in particular occur when the moisture content in the insulation space is high. The resulting decomposition products do not readily recombine, as it may occur for some decomposition products of SF₆. This is of particular relevance, since one decomposition product of the organofluorine compound is hydrogen fluoride (HF), which is highly corrosive and extremely toxic.

In order to provide for a safe operation of the apparatus, the decomposition products of the organofluorine compound shall thus be readily removed from the insulating space.

Removal of the decomposition products can theoretically be achieved by an adsorbent to which the decomposition product adsorbs and is bound permanently.

However, in particular when using a highly polar organo-fluorine compound, such as a fluoroketone, the presence of an adsorbent may lead to a decrease in the amount of organofluorine compound and to a decrease in the insulation and arc extinction performance of the insulation gas.

Considering these drawbacks, WO 2014/053661 has suggested the use of a molecular sieve, which has an average pore size y greater than the molecular size of at least one decomposition product of the organofluorine compound, and which has an adsorption capability for the organofluorine compound that is lower than for the at least one decomposition product. Thus, selective removal of the decomposition product (s) from the insulation space can be achieved, while leaving the organofluorine compound unadsorbed.

Irrespective of the achievements obtainable by the technology described in WO 2014/053661, there is nevertheless a certain risk that some water does not migrate into the material of the molecular sieve, but remains adsorbed on the outer surface of the molecular sieve. This surface-adsorbed water can react with the organofluorine compound or can give rise to a reaction cascade, which ultimately may also lead to a decrease in the insulation or arc-extinction properties.

Zeolites, for example, are able to catalyse a reaction between water and the organofluorine compound, specifically the fluoroketone, leading to the decomposition of the organo-fluorine compound, whereby heptafluoropropane, hexafluoro-propene, trifluoroacetic acid and HF are generated.

Since the vapour pressure of the organofluorine compound, in particular C5K, is rather low, it is typically to be used in combination with a carrier gas, normally represented by relatively small molecules like N₂ or CO₂.

In order to prevent the carrier gas molecules from being removed and to ensure that the composition of the insulation gas is maintained, the pore size of the zeolite should be chosen such that carrier gas molecules cannot be captured. This is difficult to achieve for small molecules; CO₂, for example, can still be adsorbed by zeolites even if they have a pore size as low as 3 A.

The invention starts from the above-mentioned WO 2014/053661 which discloses for a gas-insulated electrical apparatus to use a molecular sieve, which is for absorbing decomposition products of the organofluorine compound, in combination with a desiccant, which is different from the molecular sieve and protects the molecular sieve and the organofluorine compound against moisture. The desiccant is selected from the group consisting of: calcium, calcium sulphate, in particular drierite, calcium carbonate, calcium hydride, calcium chloride, potassium carbonate, potassium hydroxide, copper (II) sulphate, calcium oxide, magnesium, magnesium oxide, magnesium sulphate, magnesium perchlorate, sodium, sodium sulphate, aluminium, lithium aluminium hydride, aluminium oxide, activated alumina, montmorrilonite, phosphorpentoxide, silica gel, and a cellulose filter.

WO 2014/187940 discloses for an electrical apparatus having a gas-insulated chamber to provide a contamination-reduction space, which contains a molecular sieve and is separated from the gas-insulated chamber by a semipermeable membrane. The membrane is selectively premeable for at least one contaminant of the insulation gas and/or for water, but is impermeable for the components of the uncontamined insulation gas.

US 2013/0158305 A1 discloses an industrial dehumidification method for removing moisture from a fluorine-containing compound, such as hydrofluoroolefin, by bringing it into contact with a high-concentration aqueous solution containing at least one metal salt selected from the group consisting of: lithium chloride, calcium chloride, magnesium chloride, and lithium bromide. In addition to the metal salt a molecular sieve can be used to further decrease the moisture content of the fluorine-containing compound. The dehumidification is done via: bubbling gaseous fluorine-containing compound through the metal salt-containing aqueous solution; spraying the metal salt-containing aqueous solution onto the fluorine-containing compound; or flowing the fluorine-containing compound through a wet-impregnated porous body containing the metal salt-containing aqueous solution. However, in a gas-insulated electrical apparatus the use of an aqueous solution would be prohibitive, because the gas-insulated electrical apparatus requires lowest possible moisture contents to avoid corrosion of its electrical conductors and to maintain integrity and dielectric strength of its insulation gas and solid insulators.

The article by S. Bouzenada et al., “Experimental study on dehumidification/regeneration of liquid desciccant: LiBr solution”, Procedia Computer Science, Vol.32, 5 Jun. 2014, pages 673-680, discloses air conditioning based on Liquid Desiccant Cooling Systems (LDCS) using lithium bromide in aqueous solution as liquid desiccant. However, humiditiy levels in air conditioning are much higher than tolerable in electrical apparatuses. Also again, the use of aqueous solutions is per se inappropriate for gas-insulated electrical apparatuses.

The article by D. T. Acheson: “The Lithium Bromide Dew Cell” discloses atmospheric dew-point temperature measurements over an extended range of relative humidities from about 5% to 100% by using aqueous salt solution containing lithium bromide instead of lithium chloride. Around −5.5° C. the hygrometer reading shows irregularities due to abrupt water release from hydrated crystals into the aqueous lithium bromide solution. Also again, the use of aqueous solutions is per se inappropriate for gas-insulated electrical apparatuses.

The problem to be solved by the present invention is thus to provide an apparatus using an insulation gas comprising an organofluorine compound, said apparatus allowing for a relatively low dew point even at relatively high water loads and at the same time allowing for a relatively high stability of the insulation gas over time.

In particular, the present invention shall provide an apparatus using an adsorber, which is able to specifically remove water from the insulation gas and which at the same time keeps the adsorbed water in such a manner that its tendency for reaction with the organofluorine compound is reduced. More particularly, the adsorber shall have a low toxicity and be easy to handle.

The problem of the present invention is solved by the apparatus according to claim 1. Preferred embodiments of the invention are defined in the dependent claims.

According to claim 1, the invention thus relates to an apparatus for the generation, transmission, distribution and/or usage of electrical energy, said apparatus comprising a housing enclosing an insulating space and an electrical component arranged in the insulating space, said insulating space containing a dielectric insulation gas comprising an organofluorine compound.

The apparatus further comprises at least one desiccant arranged such as to come into contact with the insulation gas. According to the present invention, the desiccant contains or essentially consists of lithium bromide (LiBr).

As will be shown in further detail below, LiBr is capable of removing water in an efficient manner. The removal of water is of high relevance, not only in view of a reduced formation of decomposition products, but also in view of preventing the solid components of the electrical apparatus, in particular the moveable parts, from corroding.

By using LiBr, problems that might arise when using a molecular sieve alone, and in particular zeolite alone, can be circumvented. In particular, LiBr does neither adsorb organofluorine compound molecules nor carrier gas molecules and thus leaves the composition of the insulation gas unaffected.

Also, in comparison to a sole use of zeolite as adsorber, the tendency of decomposition of the organofluorine compound, specifically the fluoroketone, is strongly reduced when using LiBr, even in the case when the latter is in hydrated form. Without wanting to be bound by the theory, it is assumed that this is due to the adsorbed water molecules being incorporated into the salt lattice structure, thereby making the water molecules unavailable for reaction with the organofluorine compound.

In addition to the above mentioned favourable properties, LiBr is non-hazardous and does not pose any health and safety risk.

In embodiments, the desiccant consists of lithium bromide in solid, crystalline form. In other words, the desiccant does not contain an aqueous solution of lithium bromide.

In further embodiments, the desiccant does not contain a component selected from the group consisting of: calcium, calcium sulphate, in particular drierite, calcium carbonate, calcium hydride, calcium chloride, potassium carbonate, potassium hydroxide, copper (II) sulphate, calcium oxide, magnesium, magnesium oxide, magnesium sulphate, magnesium perchlorate, sodium, sodium sulphate, aluminium, lithium aluminium hydride, aluminium oxide, activated alumina, montmorrilonite, phosphorpentoxide, silica gel, and a cellulose filter.

In embodiments, the desiccant, in particular its amount and/or water loading, is chosen such that a water content of the dielectric insulation gas inside the insulating space is kept below an admissible threshold value, in particular below a threshold (or maximal) partial pressure of water vapour of 9 mbar, preferred 4 mbar, more preferred 3 mbar, most preferred 1 mbar.

Although the adsorption capacity of LiBr for water is not as high as the respective adsorption capacity of a zeolite, a dew point of −20° C. at most for a water load (i.e. with respect to the weight of lithium bromide) of up to 10% and a dew point of −10° C. for a water load (i.e. with respect to the weight of lithium bromide) of up to 40% can be achieved, which is sufficient for ensuring a safe and stable operation of the apparatus. These dew points are achieved within a relatively short period of time, namely within about 2 hours, whereas after a longer period, even lower dew points can be achieved; specifically, after 60 hours, a dew point of about −45° C. can be achieved for a water load up to 10%.

The term “water load” as used in the context of the present invention relates to ratio of the amount of water to the amount of desiccant present in the system. In this regard, the term “amount” refers to the mass of the water or the desiccant, respectively, present in the system.

In embodiments, an amount of the desiccant is dimensioned to provide a dew point inside the apparatus of below −10° C., preferred below −20° C. and most preferred −30° C.; and/or a type and amount of the molecular sieve is dimensioned to further decrease the dew point by at least 10° C., preferred 20° C. and most preferred 30° C. A combined dew point of below 30° C. or below −40° C. or below −50° C. or below −60° C. can be achieved with limited amounts of molecular sieve, such that the molecular sieve has sufficient absorption capacity for decomposition products while having little or negligible absorption capacity for the organofluorine compound, in particular fluoroketone or fluoronitrile. Such hybrid desiccant system is highly effective to reach very low dew point levels, long maintenance intervals (e.g. of at least 3 or 5 or 10 years), short time constants to reach an equilibrium state, i.e. time needed for water intake and thus dehumidification (e.g. some hours instead of some days), and combinations of such effects. In other words, the desiccant based on solid or crystalline lithium bromide allows to reduce the amount of molecular sieve to achieve very low dew points (i.e. low water content), low concentrations of decomposition products, and uncompromised concentrations of organofluorine compounds, in particular fluoroketons and fluoronitriles, in the dielectric insulation gas.

Specifically, LiBr has been found to exhibit favourable properties when being temporarily brought to elevated temperatures. This is in contrast to e.g. lithium chloride (LiCl), which is a lithium salt chemically similar to LiBr, and also in contrast to e.g. MgO₄, a salt commonly used as desiccant in many applications, since for both LiCl and MgO₄ a raise in the dew point has been observed after heating the system.

However, in the case of LiBr it has been found that although a temporary raise of the temperature leads to a raise in the dew point, the dew point again decreases after cooling to a value even lower than the initial dew point. Specifically, for a system having a dew point of −20° C., a temporary increase to a dew point of 0° C. has been observed, but the dew point decreases again upon cooling to a stable value of about −25° C., i.e. a value lower than the initial dew point. Without wanting to be bound by the theory, it is assumed that this surprising effect is due to accelerated diffusion of water from the outer layer of the desiccant towards its core, thus allowing additional water to be adsorbed.

According to embodiments, the desiccant is thus obtainable from temporarily heating a native desiccant containing or consisting of hydrated lithium bromide, specifically heating to a temperature of at least 50° C., more specifically heating to a temperature of 50° C.

In embodiments, the desiccant containing or consisting of lithium bromide (or hydrated lithium bromide) is permanently heated, in particular during operation of the apparatus for the generation, transmission, distribution and/or usage of electrical energy, to an elevated temperature, specifically heated to a temperature of at least 50° C., more specifically to a temperature of 50° C..

According to embodiments, the organofluorine compound contained in the dielectric insulation gas is selected from the group consisting of: fluoroethers (including oxiranes), in particular hydrofluoromonoethers, fluoroketones, in particular perfluoroketones, fluoroolefins, in particular hydro-fluoroolefins, fluoronitriles, in particular perfluoro-nitriles, and mixtures thereof.

It is thereby particularly preferred that the insulation gas comprises a fluoroketone containing from four to twelve carbon atoms, preferably containing exactly five carbon atoms or exactly six carbon atoms or mixtures thereof. The advantages achieved by the present invention are particularly pronounced when the insulation gas comprises a fluoroketone as defined above, since any problem, which might otherwise arise from the ketone group being subject to nucleophilic substitution, can be avoided.

The term “fluoroketone” as used in this application shall be interpreted broadly and shall encompass both perfluoroketones and hydrofluoroketones, and shall further encompass both saturated compounds and unsaturated compounds, i.e. compounds including double and/or triple bonds between carbon atoms. The at least partially fluorinated alkyl chain of the fluoro-ketones can be linear or branched, or can form a ring, which optionally is substituted by one or more alkyl groups. In exemplary embodiments, the fluoroketone is a perfluoroketone. In further exemplary embodiment, the fluoroketone has a branched alkyl chain, in particular an at least partially fluorinated alkyl chain. In still further exemplary embodiments, the fluoroketone is a fully saturated compound. As mentioned, it is particularly preferred that the insulation gas comprises a fluoroketone containing exactly five carbon atoms or exactly six carbon atoms or mixtures thereof. Compared to fluoroketones having a greater chain length with more than six carbon atoms, fluoroketones containing five or six carbon atoms have the advantage of a relatively low boiling point. Thus, problems which might go along with liquefaction can be avoided, even when the apparatus is used at low temperatures.

According to embodiments, the fluoroketone is at least one compound selected from the group consisting of the compounds defined by the following structural formulae in which at least one hydrogen atom is substituted with a fluorine atom:

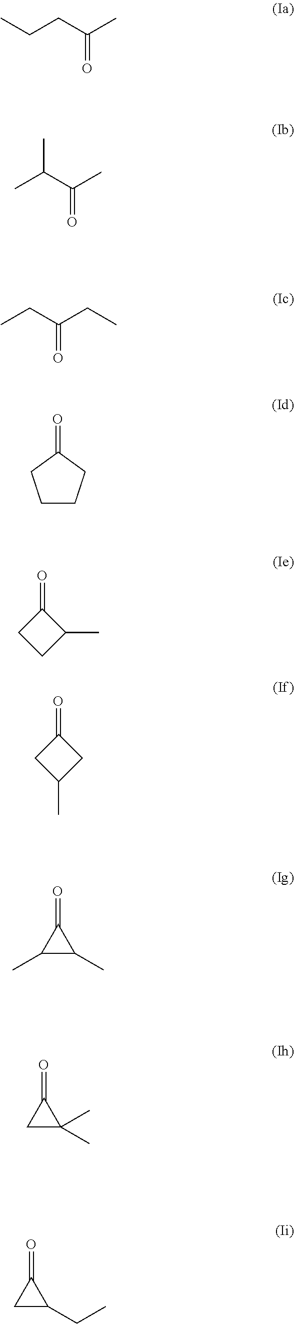

Fluoroketones containing five or more carbon atoms are further advantageous, because they are generally non-toxic with outstanding margins for human safety. This is in contrast to fluoroketones having less than four carbon atoms, such as hexafluoroacetone (or hexafluoropropanone), which are toxic and very reactive. In particular, fluoroketones containing exactly five carbon atoms, herein briefly named C5K, and fluoroketones containing exactly six carbon atoms are thermally stable up to 500° C.

In embodiments of this invention, the fluoroketones, in particular C5K, having a branched alkyl chain are preferred, because their boiling points are lower than the boiling points of the corresponding compounds (i.e. compounds with same molecular formula) having a straight alkyl chain.

According to embodiments, the C5K is a perfluoroketone, in particular has the molecular formula C₅Fi₀O, i.e. is fully saturated without double or triple bonds between carbon atoms. The fluoroketone a) may more preferably be selected from the group consisting of 1,1,1,3,4,4,4-heptafluoro-3-(trifluoromethyl) butan-2-one (also named decafluoro-2-methylbutan-3-one), 1,1,1,3,3,4,4,5,5, 5-decafluoropentan-2-one, 1,1,1,2,2,4,4,5,5,5-decafluoropentan-3-one and octafluorocylcopentanone, and most preferably is 1,1,1,3,4,4, 4-heptafluoro-3- (trifluoromethyl) butan-2-one.

1,1,1,3,4,4, 4-heptafluoro-3- (trifluoromethyl) butan-2-one can be represented by the following structural formula (I):

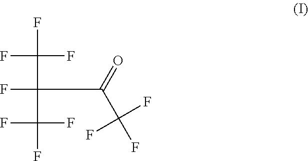 [SMILES: O=C(C(F)(F)F)C(F)(C(F)(F)F)C(F)(F)F]

1,1,1,3,4,4, 4-heptafluoro-3- (trifluoromethyl) butan-2-one with molecular formula CF₃C (0) CF (CF₃)₂ or C₅Fi₀O, has been found to be particularly preferred for high and medium voltage insulation applications, because it has the advantages of high dielectric insulation performance, in particular in mixtures with a dielectric carrier gas, has very low GWP and has a low boiling point. It has an ODP of 0 and is practically non-toxic.

According to embodiments, even higher insulation capabilities can be achieved by combining the mixture of different fluoroketone components. In embodiments, a fluoroketone containing exactly five carbon atoms, as described above and here briefly called C5K, and a fluoroketone containing exactly six carbon atoms or exactly seven carbon atoms, here briefly named fluoroketone c), can favourably be part of the dielectric insulation at the same time. Thus, an insulation gas can be achieved having more than one fluoroketone, each contributing by itself to the dielectric strength of the insulation gas.

In embodiments, the further fluoroketone c) is at least one compound selected from the group consisting of the compounds defined by the following structural formulae in which at least one hydrogen atom is substituted with a fluorine atom:

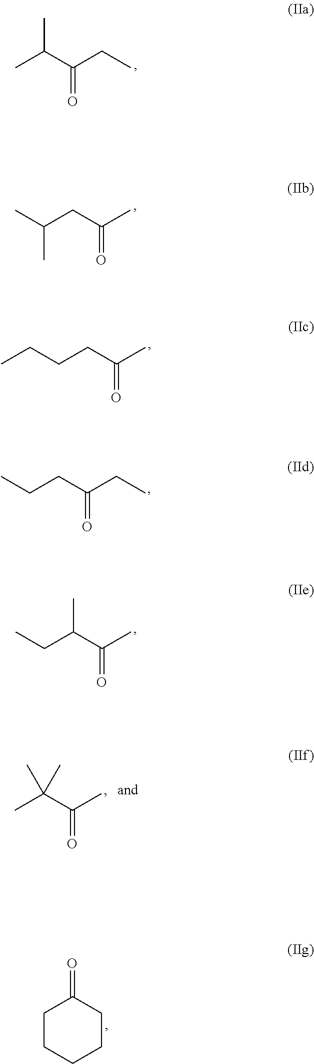

as well as any fluoroketone having exactly 6 carbon atoms, in which the at least partially fluorinated alkyl chain of the fluoroketone forms a ring, which is substituted by one or more alkyl groups (IIh);
and/or is at least one compound selected from the group consisting of the compounds defined by the following structural formulae in which at least one hydrogen atom is substituted with a fluorine atom:

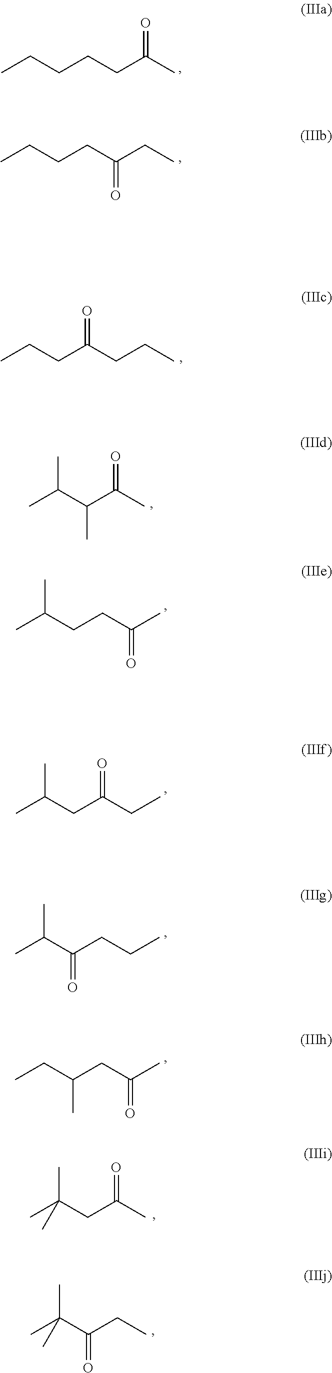

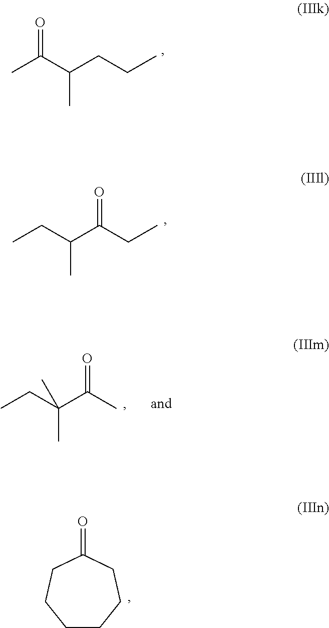

e.g. dodecafluoro-cycloheptanone, as well as any fluoroketone having exactly 7 carbon atoms, in which the at least partially fluorinated alkyl chain of the fluoroketone forms a ring, which is substituted by one or more alkyl groups (IIIo).

The present invention encompasses each compound or each combination of compounds selected from the group consisting of the compounds according to structural formulae (Ia) to (Ii), (IIa) to (IIh), (IIIa) to (IIIo), and mixtures thereof.

Depending on the specific application of the apparatus of the present invention, a fluoroketone containing exactly six carbon atoms (falling under the designation “fluoroketone c)” mentioned above) may be preferred; such a fluoroketone is non-toxic, with outstanding margins for human safety.

In embodiments, fluoroketone c), alike C5K, is a perfluoro-ketone, and/or has a branched alkyl chain, in particular an at least partially fluorinated alkyl chain; and/or the fluoroketone c) contains fully saturated compounds. E.g., the fluoroketone c) is or contains decafluorocyclohexanone. In particular, the fluoroketone c) has the molecular formula C₆FI2O, i.e. is fully saturated without double or triple bonds between carbon atoms. More preferably, the fluoroketone c) can be selected from the group consisting of: 1,1,1,2,4,4,5,5, 5-nonafluoro-2-(trifluoromethyl) pentan-3-one (also named dodecafluoro-2-methylpentan-3-one), 1,1,1,3,3,4,5,5, 5-nonafluoro-4-(trifluoromethyl) pentan-2-one (also named dodecafluoro-4-methylpentan-2-one), 1,1,1,3,4,4,5,5, 5-nonafluoro-3-(trifluoromethyl) pentan-2-one (also named dodecafluoro-3-methylpentan-2-one), 1,1,1,4,4, 4-hexafluoro-3, 3-bis-(trifluoromethyl) butan-2-one (also named dodecafluoro-3, 3-(dimethyl) butan-2-one), dodecafluorohexan-2-one, dodecafluorohexan-3-one, and particularly is the mentioned 1,1,1,2,4,4,5,5 5-Nonafluoro-2-(trifluoromethyl) pentan-3-one.

1,1,1,2,4,4,5,5, 5-Nonafluoro-2- (trifluoromethyl) pentan-3-one (also named dodecafluoro-2-methylpentan-3-one) can be represented by the following structural formula (II):

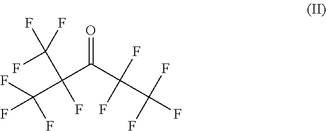 [SMILES: O=C(C(F)(F)C(F)(F)F)C(F)(C(F)(F)F)C(F)(F)F]

1,1,1,2,4,4,5,5, 5-Nonafluoro-4-(trifluoromethyl) pentan-3-one (here briefly called “C6-ketone”, with molecular formula C2F5C (0) CF (CF₃)₂) has been found to be particularly preferred for high voltage insulation applications because of its high insulating properties and its extremely low GWP. Specifically, its pressure-reduced breakdown field strength is around 240 kV/(cm*bar), which is much higher than the one of air having a much lower dielectric strength (E_{cr}=25 kV/(cm*bar). It has an ozone depletion potential of 0 and is non-toxic (LC50 of about 100000 ppm). Thus, the environmental impact is very low, and at the same time outstanding margins for human safety are achieved.

In additional or alternative embodiments, the dielectric insulation medium, in particular insulation gas, comprises at least one compound being a hydrofluoroether selected from the group consisting of: hydrofluoro monoether containing at least three carbon atoms; hydrofluoro monoether containing exactly three or exactly four carbon atoms; hydrofluoro monoether having a ratio of number of fluorine atoms to total number of fluorine and hydrogen atoms of at least 5:8; hydrofluoro monoether having a ratio of number of fluorine atoms to number of carbon atoms ranging from 1.5:1 to 2:1; pentafluoro-ethyl-methyl ether; 2,2,2-trifluoroethyl-trifluoromethyl ether; and mixtures thereof.

As mentioned above, the organofluorine compound can also be a fluoroolefin, in particular a hydrofluoroolefin. More particularly, the fluoroolefin or hydrofluorolefin, respectively, contains at least three carbon atoms or contains exactly three carbon atoms.

According to particular embodiments, the hydrofluoroolefin is thus selected from the group consisting of: 1,1,1,2-tetra-fluoropropene (HFO-1234yf; also named 2,3,3,3-tetrafluoro-1-propene), 1,2,3,3-tetrafluoro-2-propene (HFO-1234yc), 1,1,3,3-tetrafluoro-2-propene (HFO-1234zc), 1,1,1,3-tetra-fluoro-2-propene (HFO-1234ze), 1,1,2,3-tetrafluoro-2-propene (HFO-1234ye), 1,1,1,2,3-pentafluoropropene (HFO-1225ye), 1,1,2,3,3-pentafluoropropene (HFO-1225yc), 1,1,1,3,3-penta-fluoropropene (HFO-1225zc), (Z) 1,1,1,3-tetrafluoropropene (HFO-1234zeZ; also named cis-1,3,3,3-tetrafluoro-1-propene), (Z) 1,1,2,3-tetrafluoro-2-propene (HFO-1234yeZ), (E) 1,1,1,3-tetrafluoropropene (HFO-1234zeE; also named trans-1,3,3,3-tetrafluoro-1-propene), (E) 1,1,2, 3-tetrafluoro-2-propene (HFO-1234yeE), (Z) 1,1,1,2,3-pentafluoropropene (HFO-1225yeZ; also named cis-1,2,3,3,3 pentafluoroprop-1-ene), (E) 1,1,1,2,3-pentafluoropropene (HFO-1225yeE; also named trans-1,2,3,3,3 pentafluoroprop-1-ene), and mixtures thereof.

As mentioned above, the organofluorine compound can also be a fluoronitrile, in particular a perfluoronitrile. In particular, the organofluorine compound can be a fluoronitrile, specifically a perfluoronitrile, containing two carbon atoms, three carbon atoms or four carbon atoms.

More particularly, the fluoronitrile can be a perfluoro-alkylnitrile, specifically perfluoroacetonitrile, perfluoro-propionitrile (C₂F₅CN) and/or perfluorobutyronitrile (C₃F₇CN).

Most particularly, the fluoronitrile can be perfluoro-isobutyronitrile (according to the formula (CF₃)₂CFCN) and/or perfluoro-2-methoxypropanenitrile (according to the formula CF₃CF (OCF₃) CN). Of these, perfluoroisobutyronitrile is particularly preferred due to its low toxicity.

According to embodiments of the invention, the dielectric insulation gas further comprises a carrier gas. More preferably, the dielectric insulation gas comprises the organofluorine compound, particularly a fluoroketone having exactly five carbon atoms, at a partial pressure corresponding at most, in particular exactly, to the vapour pressure of the organofluorine compound at the minimum operating temperature of the apparatus, with the remainder of the dielectric insulation gas being or comprising the carrier gas. Thus, the organofluorine compound, particularly a fluoroketone having exactly five carbon atoms, is present in fully gaseous phase in the insulation space. In this regard, it is preferred that the carrier gas comprises air or an air component. In particular, the carrier gas shall be selected from the group consisting of carbon dioxide (CO₂), oxygen (O₂), nitrogen (N₂), and mixtures thereof. In particular, the carrier gas can be a mixture of N₂ and O₂, or the carrier gas can be a mixture of CO₂ and O₂. Most preferably, the carrier gas is air. Alternatively or additionally, the carrier gas can also comprise a noble gas, and/or nitric oxide, and/or nitrogen dioxide.

According to embodiments, the carrier comprises O₂, since this allows to efficiently avoid or reduce the formation of harmful decomposition products. When the carrier gas comprises O₂, the partial pressure of O₂ is preferably at least about twice that of the partial pressure of the organofluorine compound. In particular for switching applications, the carrier gas can comprise CO₂.

Although unwanted, the dielectric insulation gas typically also contains a certain amount of decomposition products. In this regard, the term “decomposition products” relates to compounds comprising less atoms than the organofluorine compound from which they are generated, i.e. the organofluorine compound of the initial composition and, particularly, the fluoroketone. In most cases, the decomposition product also has a molecular size which is substantially smaller than the molecular size of the organofluorine compound.

In addition to the desiccant containing or consisting of LiBr, the apparatus of the present invention can further comprise at least one molecular sieve arranged such as to come into contact with the insulation gas. In this regard, the molecular sieve mainly serves to adsorb or absorb decomposition products present apart from adsorbing or absorbing water.

Preferably, the pore size of the molecular sieve can be chosen such that its adsorption capability and/or absorption capability for the organofluorine compound, particularly the fluoroketone, is lower than for the decomposition product. More particularly, the pore size is chosen small enough to keep the organofluorine compound out of the pores and thus to prevent adsorption and/or absorption of the organofluorine compound to the pore surface.

In this regard, it has been found that the molecular sieve in embodiments has an average pore size y smaller than 15 A, preferably smaller than 13 A, more preferably smaller than 11 A, more preferably equal to or smaller than 9 A, preferably smaller than 7 A, more preferably smaller than 6 A and most preferably of 5 A. Specifically, a fluoroketone having five carbon atoms or more does not enter a pore of a size of smaller than 9 A and therefore does not adsorb to such a pore surface and/or is not absorbed by such a pore surface.

With regard to these embodiments, it is further preferred that the molecular sieve is at least temporarily charged with the organofluorine compound, meaning that the content of organofluorine compound in the molecular sieve is higher than its content in the dielectric insulation gas in equilibrium at operational conditions of the apparatus. In such embodiments, the organofluorine compound is not kept from entering the pores of the molecular sieve, but on the contrary is forced into the molecular sieve, in particular by exposing the molecular sieve to a gas in which the partial pressure of the organofluorine compound is higher than in the dielectric insulation gas present during operation of the apparatus. During operation of the apparatus, the organofluorine compound charging the molecular sieve is at least partially displaced by the at least one decomposition product adsorbing to the molecular sieve and/or being absorbed by the molecular sieve. The molecular sieve of this embodiment thus functions simultaneously as a “reservoir” for the organofluorine compound A as well as a “sink” for the decomposition product.

In embodiments to allow the at least one decomposition product as well as further water to be removed efficiently, the molecular sieve has an average pore y which is at least 2.7 A, preferably at least 2.8 A, more preferably at least 2.9 A, most preferably at least 3 A. It was found that this pore size is sufficient to achieve good permeation of the at least one decomposition product and water into the molecular sieve and thus good adsorption on and/or absorption by the pore surface.

In other embodiments, the molecular sieve is a zeolite, i.e. a microporous, aluminosilicate mineral that has undergone cation exchange to achieve a desired pore size. Suitable zeolites include ZEOCHEM® molecular sieve 3A (having 3 A pore size), 4A (having 4 A pore size) and 5A (having 5 A pore size).

In yet further embodiments, suitable zeolites can include e.g. ZEOCHEM® molecular sieve 23X (having a pore size of about 9 A). This can improve the “reservoir” capacity e.g. for C5-fluoroketone (C5K) while maintaining the adsorption capacity and/or absorption capacity for decomposition products, in particular since the molecular sieve is at least partly or even fully protected against water sorption by the desiccant being additionally present in the apparatus. A larger pore size of 9 A or more, in particular up to 15 A, can also be useful when larger molecules than the C5-fluoroketone are comprised in the organofluorine compound.

In embodiments, an amount of the desiccant is dimensioned to provide a dew point inside the apparatus of below −10° C., preferred below −20° C. and most preferred −30° C.; and/or a type and amount of the molecular sieve is dimensioned to further decrease the dew point by at least 10° C., preferred 20° C. and most preferred 30° C.

In embodiments, the desiccant and the molecular sieve provide in combination a dew point inside the apparatus of below −20° C., preferred below −30° C. or below −40° C., and most preferred below −50° C. or below −60 ° C.; and/or the desiccant and the molecular sieve provide in combination the dew point inside the apparatus during a maintenance interval of the apparatus, in particular during at least 1 year or at least 3 years or at least 5 years or at least 10 years.

According to further embodiments, the desiccant and/or the optionally present molecular sieve is comprised in a region of the apparatus having a temperature lower than the average temperature present in the apparatus at operational conditions. In particular, the desiccant and/or the molecular sieve can be comprised in a region to which cooling means, more particularly external cooling means, are attributed.

It is in this regard useful that the desiccant and/or the optionally present molecular sieve is or are comprised in a region of the apparatus having a temperature less than 40 K (Kelvin) above ambient temperature, more preferably less than 20 K (Kelvin) above ambient temperature.

According to an aspect of the invention and according to additional embodiments, the desiccant and/or the optionally present molecular sieve is or are in powder form. Particularly, the desiccant and/or the optionally present molecular sieve is or are designed to be at least essentially free, in particular free, of any binder in order to avoid potential issues regarding material compatibility inside the apparatus. In particular, the binder provides unwanted adsorption sites for capturing water which is then available for unwanted reaction with the organofluorine compound, in particular C5-ketone or C5-fluoroketone (C5K). Destruction of C5K molecules could then ultimately degrade the dielectric strength of the insulation gas. Therefore, leaving out the binder material can be favourable.

In additional or alternative embodiments, the desiccant and/or the optionally present molecular sieve is in the form of pellets and/or tablets.

In all aspects of the invention, it is further particularly preferred that the desiccant and/or the optionally present molecular sieve is or are comprised in a permeable container, and/or is or are arranged on a carrier, thus allowing an intensive contact between the desiccant and/or the molecular sieve, respectively, with the insulation gas. In embodiments, this permeable container or carrier can have the form of e.g. a tube, roll, fabric, lamella or honeycomb.

According to an embodiment, the at least one desiccant and/or the optionally present molecular sieve is comprised in at least one permeable container, the cover of which is permeable at least for water and more particularly is a semipermeable membrane, which is selectively permeable for water. In this embodiment, the permeable container can e.g. form a sachet.

According to an embodiment, two or more permeable containers, specifically sachets, are arranged in a frame or holder in a manner spaced apart by gaps from each other. Due to the gaps formed between the permeable containers, and thus due to their freely exposed surface area, high gas permeation into the interior of the container and thus a good contact of the desiccant and/or molecular sieve with the insulation gas can be achieved.

In a specific embodiment, the permeable containers are arranged in a cuboid frame, and more particularly are arranged parallel to each other. If the permeable container is a sachet, it is typically lined by a fabric.

According to further embodiments, the permeable containers, specifically the sachets, are flexible and are loosely arranged concentrically in a concentric holder.

The technical effects on which the present invention is based, namely the efficient removal of water without interfering with the dielectric insulation and arc extinction performance of the organofluorine-compound-comprising dielectric insulation gas, are of particular relevance for an apparatus in which the housing encloses the insulation space in a gas-tight manner. In embodiments, the apparatus is one in which the electrical component is a high voltage or medium voltage unit.

The term “transmission and distribution of electrical energy” as used in the context of the present invention encompasses the transmission or distribution of electrical energy on any voltage level.

The term “housing” as used in the context of the present invention is to be understood broadly as any at least approximately closed system. In particular, the term encompasses a plurality of chambers interconnected with each other. More particularly, “housing” encompasses a chamber, in which the electrical component is contained and which can be interconnected with a recycling system through which the dielectric insulation gas is removed, processed (e.g. cleaned) and reintroduced into the chamber. “Housing” further comprises a chamber, in which the electrical component is contained and which can be interconnected with a pre-treatment chamber for pre-treating the dielectric insulation gas prior to being introduced into the chamber.

Also, the term “arranged such as to come into contact with the insulation gas” is to be understood broadly and encompasses both embodiments where there is a permanent contact of the desiccant with the insulation gas as well as embodiments in which there is only a temporary contact of the desiccant with the insulation gas.

The term “electrical apparatus” as used in the context of the present invention specifically relates to a gas-insulated apparatus. In particular, the apparatus is part of or is or comprises a: high voltage apparatus, medium voltage apparatus, low voltage apparatus, direct-current apparatus, switchgear, air-insulated switchgear, part or component of air-insulated switchgear, gas-insulated metal-encapsulated switchgear (GIS), part or component of gas-insulated metal-encapsulated switchgear, gas-insulated line or gas-insulated transmission line (GIL), busbar, bushing, cable, gas-insulated cable, cable joint, current transformer, voltage transformer, sensor, humidity sensor, surge arrester, capacitor, inductance, resistor, insulator, air-insulated insulator, gas-insulated metal-encapsulated insulator, current limiter, high voltage switch, earthing switch, disconnector, combined disconnector and earthing switch, load-break switch, circuit breaker, gas circuit breaker, generator circuit breaker, gas-insulated vacuum circuit breaker, medium voltage switch, ring main unit, recloser, sectionalizer, low voltage switch, any type of gas-insulated switch, transformer, distribution transformer, power transformer, tap changer, transformer bushing, electrical rotating machine, generator, motor, drive, semiconducting device, computing machine, power semiconductor device, power converter, converter station, convertor building; and components and/or combinations of such devices.

In embodiments, the apparatus, in particular gas-insulated apparatus, relates to: a switchgear, in particular a gas-insulated encapsulated (e.g. metal-encapsulated) switchgear (GIS), or a part and/or component thereof.

The desiccant, and optionally the molecular sieve, can be arranged in a chamber (as part of the housing) in which the electrical component is contained, as well as in a recycling system and/or in a pre-treatment chamber (which is e.g. forming further parts of the housing).

According to embodiments, the housing comprises a chamber in which the electrical component is contained and a recycling system comprising the desiccant and the optionally present molecular sieve.

In embodiments, the desiccant is arranged in the recycling system, in particular in the chamber, such that the dielectric insulation gas in a first step comes into contact with the desiccant to reduce or eliminate moisture and only afterwards (or at least to a larger percentage or majority afterwards) in a second step comes into contact with the molecular sieve to reduce or eliminate decomposition products.

In embodiments, the recycling system is equipped with a compressor and a pump for pumping the dielectric insulation gas through at least one filter comprising the desiccant and optionally the molecular sieve. After that, the dried and cleaned dielectric insulation gas can be re-introduced into the chamber. During the operation, the humidity, density, pressure and/or content of decomposition product (s) can be measured, e.g. by gas chromatography and/or by infrared spectroscopy, and can be controlled by a respective (multi-) sensor system. An electric apparatus of such embodiments is particularly preferred as it allows cleaning and/or drying of the dielectric insulation gas without evacuation of the insulation space.

In more general terms, the housing comprises a chamber, in which the electrical component is contained, and a recycling system comprising the desiccant and/or the optionally present molecular sieve, and the housing is equipped with a compressor and a pump for pumping the dielectric insulation gas through at least one filter comprising the desiccant and/or the optionally present molecular sieve.

Apart from the apparatus described above, the present invention further relates to a method for providing a desiccant to the apparatus, the method comprising the step of temporarily heating a native desiccant containing or consisting of hydrated lithium bromide to a temperature of at least 50° C., preferably to a temperature of 50° C., and placing the desiccant thereby obtained into the insulation space of the apparatus, the heating being performed before, during or after placing the desiccant into the insulation space.

As mentioned, the heating leads to accelerated diffusion of water inside the crystal structure of the salt. When compared to a non-heated desiccant exposed to the insulation gas for the same duration, a lower dew point can be achieved by using the heated desiccant.

The term “native desiccant” refers to a fresh or thermally untreated desiccant, i.e. to the desiccant prior to the heating treatment, and differs from the untreated desiccant in its distribution of the adsorbed water: since by the heating the diffusion of water from the outer layer of the desiccant towards its core is accelerated, the amount of water in the outer layer or surface-near region is typically higher in the native desiccant than in the desiccant obtained after heating.

Throughout this application, the term “adsorbing” shall be understood broadly to encompass capturing or immobilizing molecules (as a whole or in dissociated form) on a surface of the sorbing agent (i.e. molecular sieve and/or desiccant) and can be done by any mechanism and in particular by physical or chemical binding of the molecules to the sorbing agent. In general, the term “absorbing” shall be understood broadly to encompass capturing or immobilizing molecules (as a whole or in dissociated form) in the structure of the sorbing agent (i.e. molecular sieve and/or desiccant) and can be done by any mechanism, and in particular by chemical binding the molecules (as a whole or in dissociated form) into the sorbing agent so that the absorbed molecules become part of the crystal structure of the sorbing agent.

In particular, the term “a molecular sieve” shall comprise also embodiments in which one molecular sieve or more than one molecular sieve is or are present. The term “a molecular sieve” shall thus be understood as at least one molecular sieve.

Throughout this application, terms like “preferable”, “preferred”, “advantageous”, “favourable” and the like shall designate embodiments or exemplary features only that are thus disclosed to be optional only.

The present invention is further illustrated by the following examples together with the attached

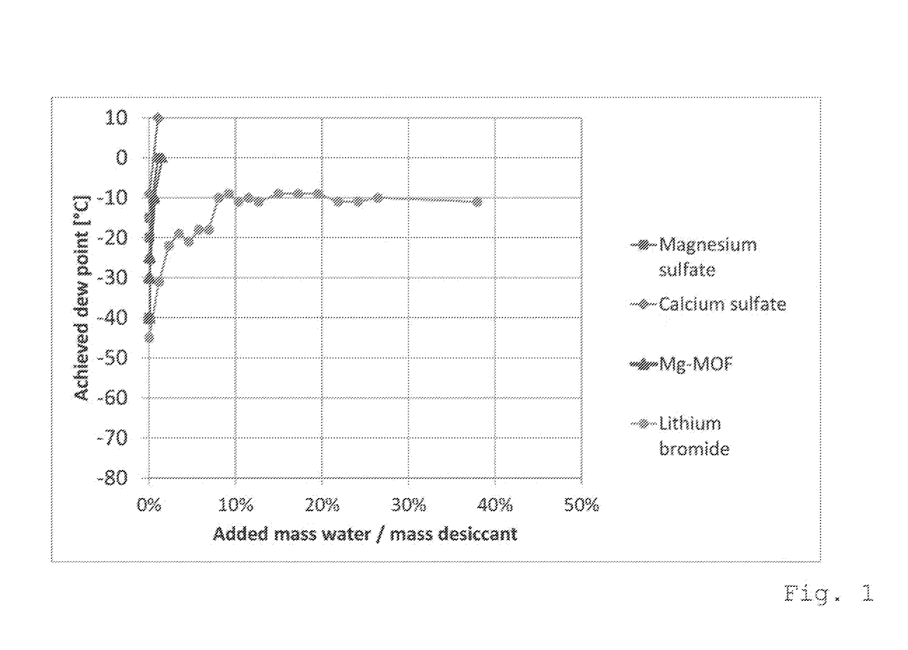

FIG. 1 showing the dew point achieved for a system comprising the desiccant according to the present invention in relation to the ratio of the mass of water to the mass of desiccant present, in comparison to other systems having different desiccants;

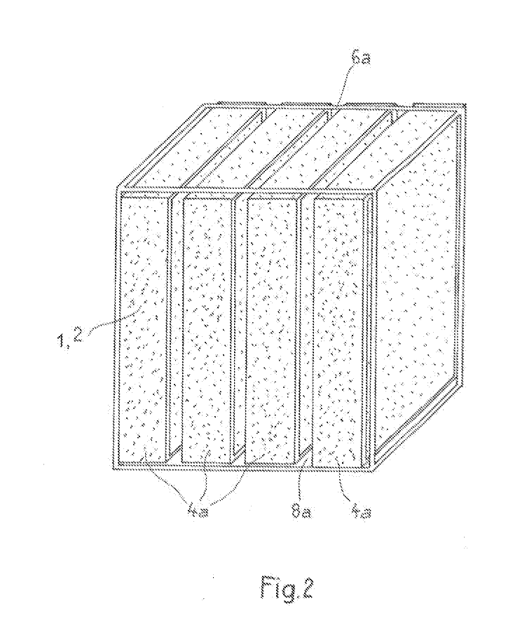

FIG. 2 showing a first arrangement of a desiccant and/or molecular sieve in powder form to be comprised in an apparatus according to the present invention;

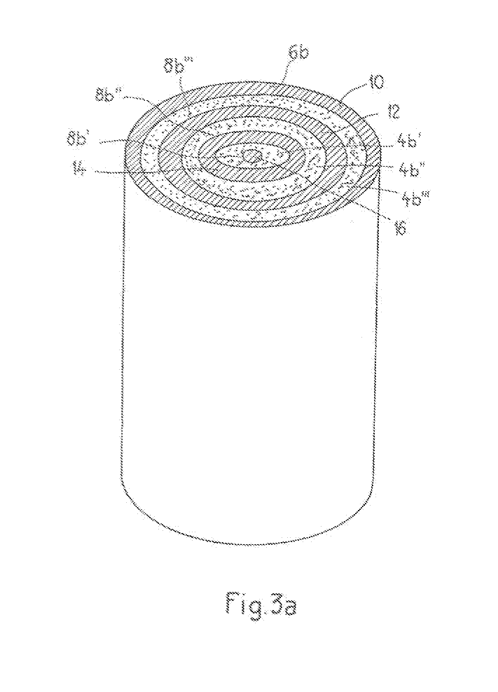

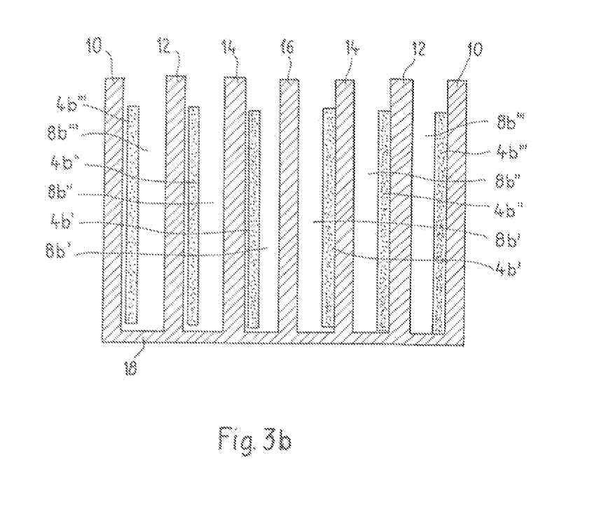

FIG. 3a showing a second arrangement of a desiccant and/or molecular sieve in powder form to be comprised in an apparatus according to the present invention in a perspective view;

FIG. 3b showing a longitudinal section of the arrangement shown in FIG. 3a; and

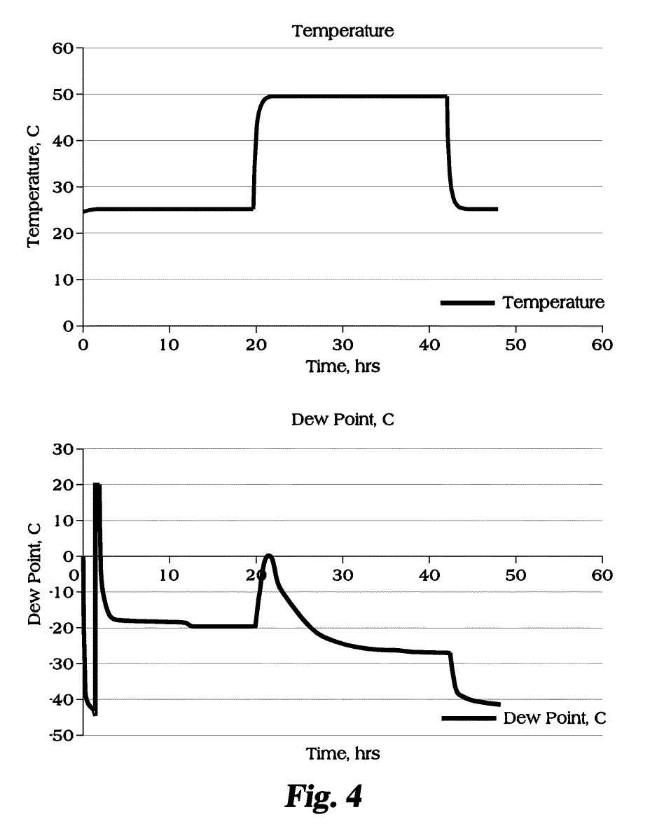

FIG. 4 showing the progress of the dew point of a system comprising the desiccant of the present invention over time, synoptically to a specific temperature profile.

As shown in FIG. 1, the use of the desiccant of the present invention, namely LiBr (presented by circles), allows to maintain a dew point of about −20° C. at a water load of up to 8% and a dew point of −10° C. at a water load of up to 40%. In contrast, the use of the comparative desiccants magnesium sulphate (squares), calcium sulphate (diamonds) and Mg-MOF (triangles) leads to a dew point of 0° C. or above already at relatively low water loads below 2%.

In the embodiments shown in FIG. 2, 3a and 3b, a desiccant and optionally a molecular sieve is or are comprised in the apparatus or test device. Two exemplary arrangements of the desiccant 1 and the optionally present molecular sieve 2 are shown in these figures.

According to the specific embodiment shown in FIG. 2, four permeable containers in the form of sachets 4a comprising the desiccant 1 and optionally the molecular sieve 2 in powder form are arranged in a frame 6a, here for example a cuboid frame 6a. The sachets 4a are arranged parallel to each other in a spaced-apart manner, such that between them a respective gap 8a is formed. Specifically, the sachets 4a are in the form of a sheet, the long sides of which corresponds more or less to the height and depth of the frame 6a, respectively. It is understood that any other form suitable for the respective purpose can be used.

According to embodiments in FIGS. 3a and 3b, the frame 6b is in cylindrical form and comprises an outermost hollow cylinder 10, in which two inner hollow cylinders 12, 14 are arranged concentrically, a middle hollow 12 cylinder and an innermost hollow cylinder 14. In the centre, a rod 16 is arranged coincidingly with the axis of the cylinders 10, 12, 14. Between the rod 16 and the innermost hollow cylinder 14, between the innermost hollow cylinder 14 and the middle hollow cylinder 12, and between the middle hollow cylinder 12 and the outermost hollow cylinder 10, radial gaps 8b′, 8b″, 8b′″, respectively, are formed. In each of the gaps 8b′, 8b41 , 8b′″, a (circumferentially rolled) sachet 4b′, 4b″, 4b′″, respectively, is arranged in a loose manner such that the surface of the sachets 4b′, 4b″, 4b′″ is not in full contact with the surface of the respective cylinders 14, 12, 10 and thus comprises a freely exposed surface area. The bottom end of the frame 6b can be closed, for example by an end plate 18, to safeguard that the desiccant 1 and optionally the molecular sieve 2 does not “fall out” of the gaps 8b′, 8b″, 8b′″.

In embodiments, any number of cylinders 14, 12, 10 can be selected to provide respective inter-cylinder spaces or gaps 8b′, 8b″, 8b′″ for receiving and holding the desiccant 1 and optionally the molecular sieve 2 in containers 4b′, 4b″, 4b′″, for example in sachets 4b′, 4b″, 4b′″, and for providing insulation-gas-accessible surface areas of the desiccant 1 and the molecular sieve 2.

FIG. 4 shows the progress of the dew point in a system, into which the desiccant lithium bromide is introduced, which immediately after introduction reduces the dew point to below −40° C. Then 5 ml of water (H₂O) is introduced into the system and leads to a temporary increase of the dew point up to 20° C. In equilibrium state the dew point reaches −20° C. As further shown in FIG. 4, a temporary raise of the temperature from about 20° C. to about 50° C. causes a temporary raise in the dew point from about −20° C. to about 0° C., because surface-near adsorbed or absorbed water is released. After this temporary increase, the dew point again decreases to a value of about −25° C. during holding the temperature at 50° C. When decreasing the temperature in the system to the initial temperature of about 25° C., the dew point even decreases to a value below −40° C., i.e. a value substantially lower than the initial equilibrium dew point of −20° C. This beneficial effect can be explained by diffusion of water from the outer layer of the desiccant lithium bromide towards its core, thus allowing additional water to be adsorbed or absorbed. An embodiment relates to a method for operating the apparatus as disclosed and claimed herein, the method comprising the step of permanently heating the desiccant containing or consisting of lithium bromide, in particular solid crystalline lithium bromide, to a temperature of at least 50° C. and preferably 50° C., when the desiccant is placed inside the insulation space of the apparatus or during operation of the apparatus.

### LIST OF REFERENCE NUMERALS

1 desiccant
2 molecular sieve
4a planar sachet
4b′, 4b″, 4b′″ rolled sachets
6a frame, cuboid frame
6b cylindrical frame
8a space, inter-planar space, planar gaps
8b′, 8b″, 8b′″ space, inter-cylinder space, radial gaps
10 outermost hollow cylinder
12 middle hollow cylinder
14 innermost hollow cylinder
16 rod
18 end plate

1. An apparatus for the generation, transmission, distribution and/or usage of electrical energy, said apparatus comprising a housing enclosing an insulating space and an electrical component arranged in the insulating space, said insulating space containing a dielectric insulation gas comprising an organofluorine compound, the apparatus further comprising a desiccant arranged such as to come into contact with the dielectric insulation gas, said desiccant consists of lithium bromide in solid, crystalline form.

2. The apparatus according to claim 1, wherein the desiccant is obtainable from temporarily heating a native desiccant containing or consisting of hydrated lithium bromide to a temperature of at least 50° C.; and/or that the desiccant is permanently heated during operation of the apparatus to an elevated temperature.

3. The apparatus according claim 1 wherein the organofluorine compound is selected from the group consisting of: fluoroethers and fluoronitriles, and mixtures thereof.

4. The apparatus according to claim 3, wherein the fluornitrile is a perfluoro-nitrile containing four carbon atoms and/or perfluoro-2-methoxypropanenitrile according to the formula CF₃CF (OCF₃) CN.

5. The apparatus according to claim 1, wherein the dielectric insulation gas comprises a fluoroketone containing from four to twelve carbon atoms.

6. The apparatus according to claim 1 wherein the dielectric insulation gas further comprises a carrier gas.

7. The apparatus according to claim 6, wherein the carrier gas comprises air or an air component.

8. The apparatus according to claim 6, wherein the dielectric insulation gas comprises the organo-fluorine compound at a partial pressure corresponding at most to the vapour pressure of the organofluorine compound at the minimum operating temperature of the apparatus, the remainder of the dielectric insulation gas being or comprising the carrier gas.

9. The apparatus according to claim 1, wherein the housing encloses the insulation space in a gas-tight manner.

10. The apparatus according to claim 1, wherein the apparatus further comprising in addition to the desiccant a molecular sieve arranged to come into contact with the insulation gas.

11. The apparatus according to claim 10, wherein the molecular sieve has an average pore size y smaller than 15 Å.

12. The apparatus according to claim 10, wherein the molecular sieve is at least temporarily charged with the organofluorine compound.

13. The apparatus according to claim 10, wherein the molecular sieve has an average pore size y which is at least 2.7 Å.

14. The apparatus according to claim 10, wherein the molecular sieve is a zeolite.

15. (canceled)

16. The apparatus according to claim 10, wherein the desiccant and the molecular sieve provide in combination the dew point inside the apparatus during a maintenance interval of the apparatus of at least 1 year.

17. The apparatus according to claim 1, wherein the electrical component is one of a high voltage apparatus or a medium voltage apparatus or a low voltage apparatus or a direct-current apparatus or a switchgear, or is a high voltage component or medium voltage component or low voltage component.

18. The apparatus according to claim 1, wherein the apparatus is a: switchgear, or part and/or component thereof, gas-insulated line (GIL), busbar, bushing, cable, gas-insulated cable, cable joint, current transformer, voltage transformer, sensor, humidity sensor, surge arrester, capacitor, inductance, resistor, insulator, air-insulated insulator, a gas-insulated metal-encapsulated Insulator, current limiter, high voltage switch, earthing switch, disconnector, combined disconnector and earthing switch, load-break switch, circuit breaker, gas circuit breaker, generator circuit breaker, gas-insulated vacuum circuit breaker, medium voltage switch, ring main unit, recloser, sectionalizer, low voltage switch, and/or any type of gas-insulated switch, transformer, distribution transformer, power transformer, tap changer, transformer bushing, electrical rotating machine, generator, motor, drive, semiconducting device, computing machine, power semiconductor device, power converter, converter station, converter building, and components and/or combinations of such devices.

19. The apparatus according to claim 1 wherein the housing comprises a chamber, in which the electrical component is contained, and a recycling system comprising the desiccant and/or the optionally present molecular sieve, and in that the housing is equipped with a compressor and a pump for pumping the dielectric insulation gas through at least one filter comprising the desiccant and optionally comprising the molecular sieve.

20. The apparatus according to claim 1, wherein the desiccant is or are comprised in a region of the apparatus having a temperature lower than the average temperature present in the apparatus at operational conditions.

21. The apparatus according to claim, wherein the desiccant is or are in the form of pellets and/or tablets.

22. The apparatus according to claim 1, wherein the desiccant is or are comprised in at least one permeable container (4a, 4b′, 4b″, 4b′″) and/or is or are arranged on at least one carrier.

23. The apparatus according to claim 1, wherein the desiccant is or are comprised in at least one permeable container (4a, 4b′, 4b″, 4b′″), the cover of which is permeable at least for water.

24. The apparatus according to claim 22, wherein two or more permeable containers (4a, 4b′, 4b″, 4b′″) are arranged in a frame or holder (6a, 6b) in a manner spaced apart by gaps (8a, 8b′, 8b″, 8b′″) from each other.

25. (canceled)

26. The apparatus according to claim 1, wherein the desiccant does not contain an aqueous solution of lithium bromide.

27. The apparatus according to claim 1, wherein the desiccant does not contain a component selected from the group consisting of: calcium, calcium sulphate, in particular drierite, calcium carbonate, calcium hydride, calcium chloride, potassium carbonate, potassium hydroxide, copper(II) sulphate, calcium oxide, magnesium, magnesium oxide, magnesium sulphate, magnesium perchlorate, sodium, sodium sulphate, aluminium, lithium aluminium hydride, aluminium oxide, activated alumina, montmorrilonite, phosphorpentoxide, silica gel, and a cellulose filter.

28. The apparatus accord to claim 1, wherein the desiccant is chosen such that a water content of the dielectric insulation gas inside the insulating space is kept below an admissible threshold value, in particular below a threshold partial pressure of water vapour of 9 mbar.

29. A method for providing a desiccant to an apparatus for the generation, transmission, distribution and/or usage of electrical energy, the apparatus comprising a housing enclosing an insulating space and an electrical component arranged in the insulating space, the insulating space containing a dielectric insulation gas comprising an organofluorine compound, the apparatus further comprising a desiccant arranged such as to come into contact with the dielectric insulation gas, the desiccant consists of lithium bromide in solid, crystalline form, the method comprising:
temporarily heating a native desiccant consisting of hydrated lithium bromide to a temperature of at least 50° C.;
placing the desiccant thereby obtained into the insulation space of the apparatus, the heating being performed before, during or after placing the desiccant into the insulation space,

30. A method for operating an apparatus for the generation, transmission, distribution and/or usage of electrical energy, the apparatus comprising a housing enclosing an insulating space and an electrical component arranged in the insulating space, the insulating space containing a dielectric insulation gas comprising an organofluorine compound, the apparatus further comprising a desiccant arranged such as to come into contact with the dielectric insulation gas, the desiccant consists of lithium bromide in solid, crystalline form, the method comprising:
permanently heating the desiccant consisting of the solid crystalline lithium bromide, to a temperature of at least 50° C. when being placed inside the insulation space of the apparatus or during operation of the apparatus.
